(19) United States
(12) Patent Application Publication
(10) Pub. No.: US 2023/0372604 A1
(43) Pub. Date: Nov. 23, 2023

(54) Med-Wire Device

(71) Applicant: Carla Nugent, Concord, NC (US)

(72) Inventor: Carla Nugent, Concord, NC (US)

(21) Appl. No.: 18/317,218

(22) Filed: May 15, 2023

Related U.S. Application Data
(60) Provisional application No. 63/343,691, filed on May 19, 2022.

(51) Int. Cl. A61M 5/14

(52) U.S. Cl. CPC A61M 5/1418

(57) ABSTRACT

The present invention relates to a novel medical wire device. The device is designed to prevent the bundling and tangling of cords in a medical setting, which may result in a patient tripping and falling. The device is comprised of a transparent and flexible housing. Cords and wires are held inside via small slots within the housing. A soft closed snap mechanism is used to secure the cords and wires within the housing when the device is in a closed position. Thus, users can independently store and organize the cords to prevent them from tangling together and causing trips and falls.

### CROSS-REFERENCE TO RELATED APPLICATION

The present application claims priority to, and the benefit of, U.S. Provisional Application No. 63/343,691, which was filed on May 19, 2022, and is incorporated herein by reference in its entirety.

### FIELD OF THE INVENTION

The present invention relates generally to the field of medical wire devices. More specifically, the present invention relates to an improved medical wire device that provides users with a small device designed to secure medical wires and cords to prevent tangling. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in medical wire devices for detangling and organizing medical wires and cords. In medical settings where a patient is connected to several different monitors and machines, cord tangling can be a significant problem. Wires can tangle, and a patient could trip and fall. Falls for those with preexisting injuries and/or the elderly can cause serious complications in their treatment. Furthermore, nurses and other staff may spend more time untangling cords than treating a patient.

Additionally, many types of surgeries require a multiplicity of tubes and/or electric wires and cords to be inserted into the patient or attached to electrodes on the patient. Problems have arisen in the past in providing adequate means for securing such tubes and cords so that they may be firmly held during the operation in positions in which they will not interfere with the surgeon, anesthetist, or attendant nurses.

Thus, it is necessary for users to have a device which retains such medical cords and wires and prevents them from becoming tangled. The improved medical wire device of the present invention, as will become more evident as the present description proceeds, finds particular utility in anchoring and organizing such medical tubes and cords during surgical operations and/or post-operative care, etc.

Therefore, there exists a long-felt need in the art for a medical wire device that provides users with a small, plastic device designed to secure medical wires and cords to prevent tangling. There is also a long-felt need in the art for a medical wire device that features two or more slots in each device that hold cords and wires independently, ensuring they cannot tangle and mix together. Further, there is a long-felt need in the art for a medical wire device that allows users to secure the cords and wires in place via a soft snap mechanism on one side of the device. Moreover, there is a long-felt need in the art for a device that enables users to wash and reuse the device as needed to accommodate several different patients in a hospital or other medical setting. Further, there is a long-felt need in the art for a medical wire device that offers a way to reduce the chance of trips, falls, and injuries occurring due to loose cords and wires. Finally, there is a long-felt need in the art for a medical wire device that is reusable and capable of being sanitized.

The subject matter disclosed and claimed herein, in one embodiment thereof, comprises a medical wire device. The device is designed to prevent the bundling and tangling of cords in a medical setting which may result in a patient tripping and falling. The device is comprised of a transparent and flexible housing. The cords and wires are held inside via small slots within the housing. A soft closed snap mechanism is used to secure the cords and wires within the housing when the device is in a closed position. Any suitable cords can be retained within the device, such as telemetry cords, intravenous lines, and oxygen tubing, etc. Users can independently store and organize the cords to prevent them from tangling together and causing trips and falls. The device is reusable and capable of being sanitized.

In this manner, the medical wire device of the present invention accomplishes all of the foregoing objectives and provides users with a device that secures wires and cords to prevent tangling. The device allows a user to sterilize and reuse the device, if necessary. The device reduces the chance of trips, falls, and injuries for patients and other users, due to loose cords and wires.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a medical wire device. The device is designed to prevent the bundling and tangling of cords in a medical setting which may result in a patient tripping and falling. The device is comprised of a transparent and flexible housing. The cords and wires are held inside via small slots within the housing. A soft closed snap mechanism is used to secure the cords and wires within the housing when the device is in a closed position. Users can independently store and organize the cords to prevent them from tangling together and causing trips and falls.

In one embodiment, the housing of the medical wire device is essentially hollow and comprises an internal cavity. In this embodiment, the housing comprises two identical halves which are secured together. The housing and/or the housing halves are typically shaped as a trapezoid but can be any suitable shape as is known in the art, as long as the housing can retain multiple cords and/or wires within it. Further, the housing halves are secured at one side via at least one hinge, or other movable joint which allows the housing halves to open and close.

In another embodiment, the opposing side of the housing halves comprises a securing mechanism, such as a soft close snap mechanism, or other suitable securing mechanism as is known in the art. Any securing means can be used, as long as the securing mechanism allows the housing halves to be releasably secured together, such that a user can open and close the housing halves.

In yet another embodiment, the housing halves comprise a plurality of small slots or ridges within the inside of the housing. Any number of slots or ridges can be utilized depending on the needs and/or wants of a user. Each medical cable, wire, cord, etc., would be individually placed within one slot. Once the desired number of slots or ridges are filled with cables or wires, the housing halves are closed and secured. The slots can be any suitable size as is known in the art, as long as they are large enough to retain a wire or cable.

In one embodiment, the slots or ridges form semi-circle shapes for accepting cords and wires. Specifically, each housing half comprises at least one semi-circle shape which mates with an identical semi-circle shape from the second housing half. Thus, when the housing halves are secured together, the semi-circle shapes mate forming a complete circle for retaining a cord or wire. The semi-circle shapes and the complete circle shapes can be any suitable size as is known in the art, as long as they are large enough to retain a wire or cable. In this embodiment, the semi-circle shapes are positioned at the front edges or perimeter of the housing or at the rear or back edges or perimeter of the housing. In one embodiment, the semi-circle shapes are positioned at both the front edges and the back edges of the housing.

In one embodiment, the medical wire device comprises a securing means on at least one side of the housing. In one embodiment, the medical wire device comprises a securing means on both sides of the housing. The securing means can be any suitable securing means as is known in the art, such as a thin flexible fabric pad which may comprise hook and loop fasteners, a pressure-sensitive adhesive layer with an appropriate backing paper, a clamp, or any other suitable securing means as is known in the art which engages a surface to retain the device in position.

In one embodiment, the medical wire device is reusable and capable of being sanitized. Typically, the medical wire device is manufactured of a material that allows the device to be washed and reused, and/or sanitized for reuse, depending on the needs and/or wants of a user.

In yet another embodiment, the medical wire device is manufactured from heat-sealable plastic or polymers, such as polypropylene or acrylonitrile-butadiene-styrene (ABS), polyethylene, polystyrene, etc.

In yet another embodiment, the medical wire device is manufactured from a material that is water resistant.

In yet another embodiment, the medical wire device comprises a plurality of indicia.

In yet another embodiment, a method of securing medical wires and cords to prevent tangling is described. The method includes the steps of providing a medical wire device comprising a housing with small slots for retaining the cords and wires and a snap closure for securely closing the device. The method also comprises positioning the cords and wires within the small slots, such that each wire or cord is placed in its own slot. Further, the method comprises securing the cords and wires within the device via the snap closure by snapping the housing together. Finally, positioning the device in a desired location without worry of tangling cords and wires, thus preventing falls and injuries.

In yet another embodiment of the present invention, the medical wire device is disclosed for independently storing and organizing a plurality of cords and/or wires to prevent them from tangling together and causing trips and falls. The device comprises a housing that opens into two identical halves which are secured together via a hinge or other means. Cords and/or wires are placed within the housing half, such that the cords and/or wires are each positioned within a slot or semi-circular shaped opening. Once the desired number of cords and/or wires are positioned within the housing, the housing is closed and secured via a snap mechanism or other securing means. The device can then be placed in any desired position or can also be secured to a surface via an attached adhesive or other securing means. After use, the device can be washed and/or sanitized and reused as needed. Any suitable cords and/or wires can be utilized with the device, such as telemetry cords, intravenous lines, oxygen tubing, etc., or any other suitable cords and/or wires as is known in the art.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the medical wire device of the present invention in accordance with the disclosed architecture;

FIG. 2 illustrates a perspective view of one embodiment of the medical wire device of the present invention in an open position in accordance with the disclosed architecture;

FIG. 3 illustrates a perspective view of one embodiment of the medical wire device of the present invention showing multiple different types of cords and wires in accordance with the disclosed architecture;

FIG. 4 illustrates a perspective view of one embodiment of the medical wire device of the present invention disclosing the cord and wire slots in accordance with the disclosed architecture;

FIG. 5 illustrates a perspective view of one embodiment of the medical wire device of the present invention in use in accordance with the disclosed architecture; and

FIG. 6 illustrates a flowchart showing the method of securing medical wires and cords to prevent tangling in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a medical wire device that provides users with a small, plastic device designed to secure medical wires and cords to prevent tangling. There is also a long-felt need in the art for a medical wire device that features two or more slots in each device that hold cords and wires independently, ensuring they cannot tangle and mix together. Further, there is a long-felt need in the art for a medical wire device that allows users to secure the cords and wires in place via a soft snap mechanism on one side of the device. Moreover, there is a long-felt need in the art for a device that enables users to wash and reuse the device as needed to accommodate several different patients in a hospital or other medical setting. Further, there is a long-felt need in the art for a medical wire device that offers a way to reduce the chance of trips, falls, and injuries occurring due to loose cords and wires. Finally, there is a long-felt need in the art for a medical wire device that is reusable and capable of being sanitized.

The present invention, in one exemplary embodiment, is a novel medical wire device. The device is designed to prevent the bundling and tangling of cords in a medical setting which may result in a patient tripping and falling. The device is comprised of a transparent and flexible housing. The cords and wires are held inside via small slots within the housing. A soft closed snap mechanism is used to secure the cords and wires within the housing when the device is in a closed position. Users can independently store and organize the cords to prevent them from tangling together and causing trips and falls. The present invention also includes a novel method of securing medical wires and cords to prevent tangling. The method includes the steps of providing a medical wire device comprising a housing with small slots for retaining the cords and wires and a snap closure for securely closing the device. The method also comprises positioning the cords and wires within the small slots, such that each wire or cord is placed in its own slot. Further, the method comprises securing the cords and wires within the device via the snap closure by snapping the housing together. Finally, positioning the device in a desired location without worry of tangling cords and wires, thus preventing falls and injuries.

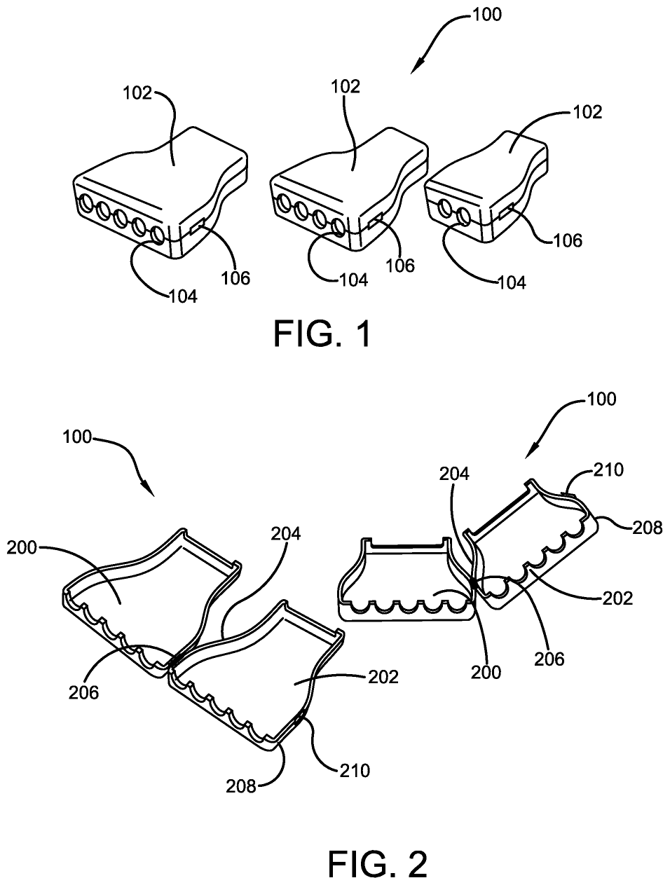

Referring initially to the drawings, FIG. 1 illustrates a perspective view of one embodiment of the medical wire device 100 of the present invention. In the present embodiment, the medical wire device 100 is an improved medical wire device that secures wires and cords and prevents tangling and bundling of the cords in a medical setting which may result in a patient tripping and falling. The device 100 is especially designed to allow patients, doctors, nurses, lab technicians, etc., or any other suitable user as is known in the art, to easily maintain multiple cords and wires without worry of tangling and causing injuries. More specifically, the device 100 comprises a housing 102 with a plurality of small slots 104 for retaining cords and wires 300. The housing 102 comprises a snap closure 106 which then secures the housing 102 together and secures the cords and wires 300 within the housing 102. The device 100 can then be placed in a desired location without worry of cords and wires 300 tangling and causing accidents and injuries. Thus, the device 100 allows users to independently store and organize cords and/or wires 300 to prevent them from tangling together and causing trips and falls.

As shown in FIGS. 1-2, the housing 102 of the medical wire device 100 is essentially hollow and comprises an internal cavity for retaining the cords and/or wires 300. In this embodiment, the housing 102 comprises two identical halves, (i.e., a first half 200 and a second half 202), which are secured together. The housing 102 and/or the housing halves 200, 202 are typically trapezoidal in shape, but can be any suitable shape as is known in the art, as long as the housing 102 can retain multiple cords and/or wires 300 within it. Further, the housing halves 200, 202 are secured at one side 204 via at least one hinge 206, or other movable joint which allows the housing halves 200, 202 to open and close. Any suitable number of hinges 206 can be utilized as is known in the art, depending on the needs and/or wants of a user.

Furthermore, the opposing side, (i.e., the second side 208) of the housing halves 200, 202 comprises a securing mechanism 210, such as a soft close snap mechanism 106, or other suitable securing mechanism as is known in the art. Any suitable securing means 210 can be used, as long as the securing mechanism 210 allows the housing halves 200, 202 to be releasably secured together, such that a user can open and close the housing halves 200, 202. In one embodiment, the housing halves 200, 202 do not comprise a hinge 206 but instead comprise a securing means 210 on both sides 204, 208, such that the housing halves 200, 202 are releasably secured together. In this embodiment, the securing means 210 can be released, separating both housing halves 200, 202 into two distinct halves 200 202. Once, the cords and/or wires 300 are positioned within the housing 102, the housing halves 200, 202 are then secured together via the securing means 210.

Furthermore, the housing halves 200, 202 comprise a plurality of small slots or ridges 104 within the inside of the housing 102. Any suitable number of slots or ridges 104 can be utilized depending on the needs and/or wants of a user. Each medical cable, wire, cord, etc. 300, would be individually placed within one slot 104. Once the desired number of slots or ridges 104 are filled with the desired cables or wires 300, the housing halves 200, 202 are closed and secured. The slots 104 can be any suitable size as is known in the art, as long as they are large enough to retain a wire or cable 300.

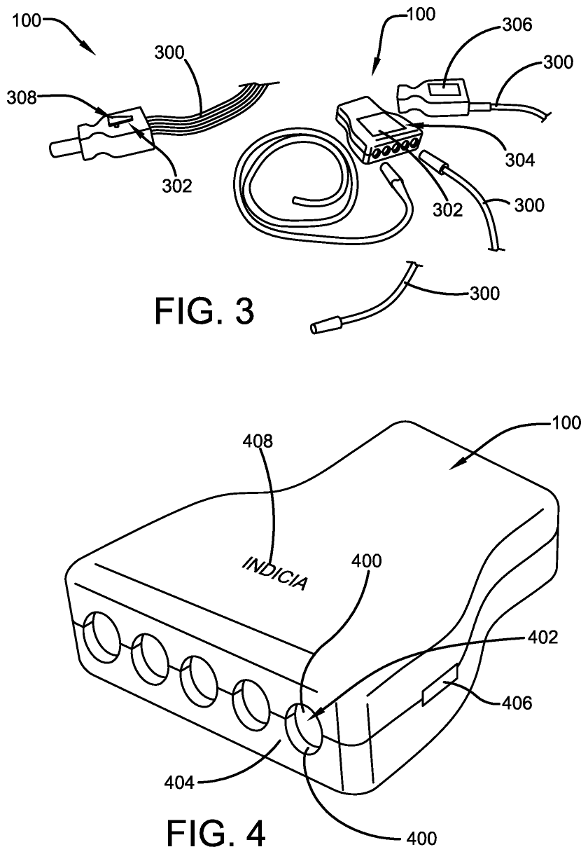

As shown in FIG. 3, the medical wire device 100 comprises a securing means 302 on at least one side 204, 208 of the housing 102. In one embodiment, the medical wire device 100 comprises a securing means 302 on both sides 204, 208 of the housing 102. The securing means 302 can be any suitable securing means 302 as is known in the art, such as a thin flexible fabric pad which may comprise hook and loop fasteners 304, a pressure-sensitive adhesive layer with an appropriate backing paper 306, a clamp 308, or any other suitable securing means 302 as is known in the art which engages a surface to retain the device 100 in position.

Additionally, any suitable type of wires and/or cables 300 can be utilized with the device 100. For example, telemetry cords, intravenous lines, and/or oxygen tubing, etc., or any other suitable wires and/or cables 300 as is known in the art can be used, depending on the needs and/or wants of a user.

As shown in FIG. 4, the slots or ridges 104 of the device 100 can form semi-circle shapes 400 for accepting cords and wires 300. Specifically, each housing half 200 comprises at least one semi-circle shape 400 which mates with an identical semi-circle shape 400 from the second housing half 202. Thus, when the housing halves 200, 202 are secured together, the semi-circle shapes 400 mate forming a complete circle 402 for retaining a cord or wire 300. The semi-circle shapes 400 and the complete circle shapes 402 can be any suitable size as is known in the art, as long as they are large enough to retain a wire or cable 300. In this embodiment, the semi-circle shapes 400 are positioned at the front edges 404 or perimeter of the housing 102 or at the rear or back edges 406 or perimeter of the housing 102. In one embodiment, the semi-circle shapes 400 are positioned at both the front edges 404 and the back edges 406 of the housing 102.

Furthermore, the medical wire device 100 is reusable and capable of being sanitized. Typically, the medical wire device 100 will be manufactured of a material that allows the device 100 to be washed and reused, and/or sanitized for reuse, depending on the needs and/or wants of a user. Specifically, the medical wire device 100 is typically manufactured from heat-sealable plastic or polymers, such as polypropylene or acrylonitrile-butadiene-styrene (ABS), or any other suitable material as is known in the art, such as, but not limited to, polycarbonate, polyethylene, polyethylene terephthalate, polyvinyl chloride, polystyrene, etc. Generally, the medical wire device 100 is also manufactured from a material that is water resistant or water permeable, or the housing 102 comprises a coating that is water resistant or water permeable.

In another embodiment, the medical wire device comprises a plurality of indicia 408. The housing 102 of the device 100 may include advertising, a trademark, or other letters, designs, or characters, printed, painted, stamped, or integrated into the housing 102, or any other indicia 408 as is known in the art. Specifically, any suitable indicia 408 as is known in the art can be included, such as, but not limited to, patterns, logos, emblems, images, symbols, designs, letters, words, characters, animals, advertisements, brands, etc., that may or may not be medical or brand related.

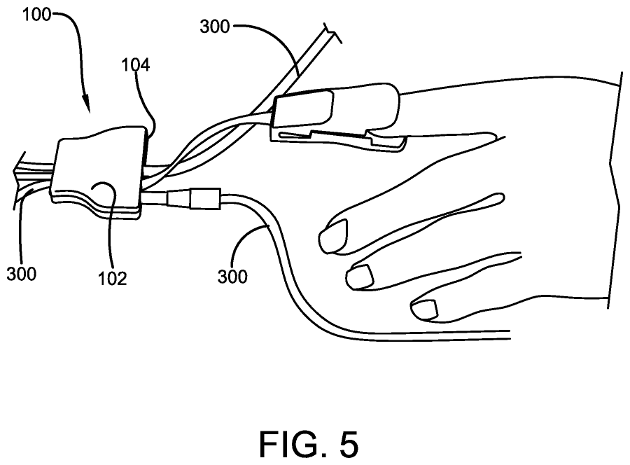

As shown in FIG. 5, the medical wire device 100 is disclosed for independently storing and organizing a plurality of cords and/or wires 300 to prevent them from tangling together and causing trips and falls. The device 100 comprises a housing 102 that opens into two identical halves 200, 202 which are secured together via a hinge 206 or other means. Cords and/or wires 300 are placed within the housing half 200, 202, such that the cords and/or wires 300 are each positioned within a slot 104 or semi-circular shaped opening 400. Once the desired number of cords and/or wires 300 are positioned within the housing 102, the housing 102 is closed and secured via a snap mechanism 106 or other securing means 210. The device 100 can then be placed in any desired position or can also be secured to a surface via an attached adhesive 306 or other securing means 302. After use, the device 100 can be washed and/or sanitized and reused as needed. Any suitable cords and/or wires 300 can be utilized with the device 100, such as telemetry cords, intravenous lines, oxygen tubing, etc., or any other suitable cords and/or wires 300 as is known in the art.

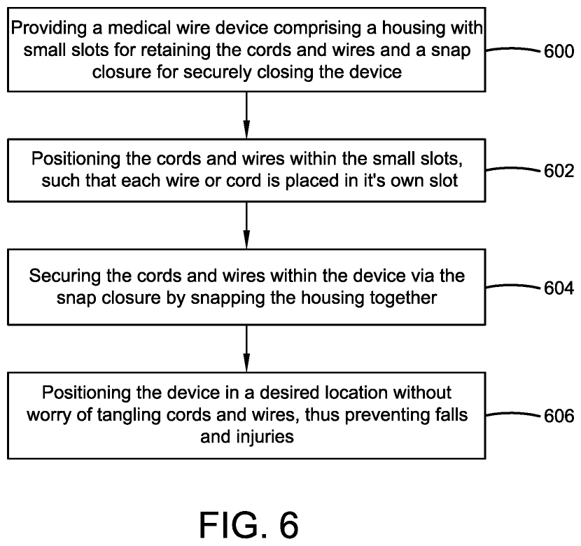

FIG. 6 illustrates a flowchart of the method of securing medical wires and cords to prevent tangling. The method includes the steps of at 600, providing a medical wire device comprising a housing with small slots for retaining the cords and wires and a snap closure for securely closing the device. The method also comprises at 602, positioning the cords and wires within the small slots, such that each wire or cord is placed in its own slot. A user opens the housing, separating the halves and places the cords and/or wires into the slots along one of the halves. Once in place, the user secures the second half to the first half, securing the housing together. Further, the method comprises at 604, securing the cords and wires within the device via the snap closure by snapping the housing together. Finally, at 606, positioning the device in a desired location without worry of tangling cords and wires, thus preventing falls and injuries. The device can be positioned in any desired location and can even be secured to a surface via an attached adhesive or other securing means.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “medical wire device”, “wire device”, and “device” are interchangeable and refer to the medical wire device 100 of the present invention.

Notwithstanding the foregoing, the medical wire device 100 of the present invention can be of any suitable size and configuration as is known in the art without affecting the overall concept of the invention, provided that it accomplishes the above-stated objectives. One of ordinary skill in the art will appreciate that the medical wire device 100 as shown in FIGS. 1-6 are for illustrative purposes only, and that many other sizes and shapes of the medical wire device 100 are well within the scope of the present disclosure. Although the dimensions of the medical wire device 100 are important design parameters for user convenience, the medical wire device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical wire device that secures a plurality of wires and cords and prevents tangling and bundling of the plurality of wires and cords in a medical setting, the medical wire device comprising:
a housing;
a plurality of slots that are sized and configured to retain the plurality of wires and cords; and
an internal cavity for retaining the plurality of slots, wherein each of the plurality of wires and cords is individually placed within one of the plurality of slots, and further wherein the housing is then placed in a desired location without worry of the plurality of wires and cords becoming tangled or causing an accident or injury.

2. The medical wire device of claim 1, wherein the housing is trapezoidal in shape.

3. The medical wire device of claim 2, wherein the housing comprises a first half and a second half and further wherein the first and second halves are identical.

4. The medical wire device of claim 3, wherein the first half and the second half are secured on a first side via a hinge.

5. The medical wire device of claim 4, wherein the first half and the second half are secured on a second side via a securing mechanism.

6. The medical wire device of claim 5, wherein the securing mechanism is a snap mechanism.

7. The medical wire device of claim 1, wherein the housing comprises a securing means on a first side for securing the medical wire device to a surface.

8. The medical wire device of claim 7, wherein the securing means is a hook and loop fastener.

9. The medical wire device of claim 7, wherein the securing means is a pressure-sensitive adhesive layer with a backing paper.

10. The medical wire device of claim 7, wherein the securing means is a clamp.

11. The medical wire device of claim 1, wherein the plurality of wires and cords can be a telemetry cord, an intravenous line, an oxygen tube or a combination thereof.

12. The medical wire device of claim 3, wherein the plurality of slots form a semi-circle shape for accepting the plurality of wires and cords.

13. The medical wire device of claim 12, wherein the first half of the housing comprises a plurality of semi-circle shapes that mate with a second plurality of semi-circle shapes on the second half of the housing to form a complete circle for retaining the plurality of wires and cords.

14. The medical wire device of claim 1, wherein the medical wire device is comprised of a material that is reusable and capable of being sanitized.

15. A medical wire device that secures a plurality of wires and cords and prevents tangling and bundling of the plurality of wires and cords in a medical setting, the medical wire device comprising:
a housing comprising a first half and a second half;
wherein the first half and the second half are secured on a first side via a hinge;
wherein the first half and the second half are secured on a second side via a snap mechanism;
wherein the first half comprises a plurality of semi-circle shapes along a front edge which mate with a plurality of semi-circle shapes on a front edge of the second half to form a complete circle for retaining the plurality of wires and cords;
wherein each of the plurality of wires and cords are individually placed within one of the plurality of semi-circle shapes; and
wherein the first half and the second half of the housing are then secured together via the snap mechanism, thereby retaining the plurality of wires and cords inside.

16. The medical wire device of claim 15 further comprising a plurality of indicia.

17. The medical wire device of claim 15, wherein the medical wire device is comprised of a material that is reusable and capable of being sanitized.

18. The medical wire device of claim 15, wherein the housing comprises a securing means on a first side for securing the medical wire device to a surface.

19. The medical wire device of claim 18, wherein the securing means is a hook and loop fastener, a pressure-sensitive adhesive layer with a backing paper, or a clamp.

20. A method of securing a plurality of medical wires and cords to prevent tangling, the method comprising the steps of:
providing a medical wire device comprising a housing having a plurality of slots for retaining the plurality of medical wires and cords and a snap closure for securely closing the medical wire device;
positioning the plurality of medical wires and cords within the plurality of slots, such that each of the plurality of medical wires and cords is placed in its own slot of the plurality of slots;
securing the plurality of medical wired and cords within the medical wire device via the snap closure by snapping the housing together; and
positioning the medical device in a desired location without worry of tangling the plurality of medical wires and cords.
